(19) United States
(12) Patent Application Publication
(10) Pub. No.: US 2026/0041310 A1
(43) Pub. Date: Feb. 12, 2026

(54) ARTICULATING MEDICAL DEVICES AND RELATED METHODS

(71) Applicant: Boston Scientific Scimed, Inc., Maple Grove, MN (US)

(72) Inventors: Brent DOLAN, Blaine, MN (US); Daniel J. FOSTER, Lino Lakes, MN (US); Bradley S. SWEHLA, Eagan, MN (US); Eric T. GAGNER, Minnetonka, MN (US)

(73) Assignee: Boston Scientific Scimed, Inc., Maple Grove, MN (US)

(21) Appl. No.: 19/288,022

(22) Filed: Aug. 1, 2025

Related U.S. Application Data
(60) Provisional application No. 63/681,301, filed on Aug. 9, 2024.

(51) Int. Cl. A61B 1/005; A61B 1/00; A61B 1/018; A61B 1/05; A61B 1/06; A61B 90/00

(52) U.S. Cl. CPC A61B 1/0055; A61B 1/00128; A61B 1/0052; A61B 1/0057; A61B 1/018; A61B 1/05; A61B 1/0676; A61B 90/39; A61B 2090/3966

(57) ABSTRACT

Medical devices with articulation members are described, including a medical device that includes a shaft defining a working channel extending from a proximal end to a distal portion of the shaft. The distal portion of the shaft includes an articulation member comprising a plurality of segments radially outward of the working channel. Each segment may have an arc-shaped cross-section and define at least one aperture, e.g., aligned with the aperture(s) an adjacent segment for receiving a steering wire therethrough. Each segment is pivotable relative to the adjacent segment. The articulation member also includes a rib extending along a longitudinal axis of the shaft and coupled to, or integral with, each segment of the plurality of segments. The rib extends radially outward from the plurality of segments in a direction away from the working channel.

### CROSS-REFERENCE TO RELATED APPLICATIONS

This application claims the benefit of priority to U.S. Provisional Application No. 63/681,301, filed on Aug. 9, 2024, which is incorporated by reference herein in its entirety.

### TECHNICAL FIELD

Aspects of the disclosure generally relate to articulating medical devices, systems, and methods. Some aspects relate to medical devices including an articulation member and methods of use thereof.

### BACKGROUND

During a medical procedure, a user may operate a medical device with a handle and a shaft extending distally from the handle. Some medical device shafts feature an articulation member, which takes up space within the shaft that might otherwise be useful for transporting tools, providing irrigation or suction at a target site of a patient, and/or housing internal componentry and electrical wires. As medical devices evolve to provide for articulation, the inner diameter of the shaft may become constrained, limiting the space available for aforementioned items including medical tools such as forceps, needles, and brushes.

### SUMMARY

The disclosure includes medical devices with articulation members providing relatively more space to accommodate, for example, medical tools and connections to electronic components of medical devices.

For example, the disclosure includes a medical device comprising a shaft defining a working channel extending from a proximal end to a distal portion of the shaft. The distal portion of the shaft may include an articulation member comprising a plurality of segments radially outward of the working channel. Each segment may have an arc-shaped cross-section and define at least one aperture. The at least one aperture of each segment may be aligned with the at least one aperture of an adjacent segment for receiving a steering wire therethrough. In addition, each segment may be pivotable relative to the adjacent segment. The articulation member also may include a rib extending along a longitudinal axis of the shaft and coupled to, or integral with, each segment of the plurality of segments. The rib may extend radially outward from the plurality of segments in a direction away from the working channel.

According to some aspects, the rib may be coupled to the plurality of segments and/or the rib and the plurality of segments may comprise different materials. The rib may comprise a metal or metal alloy. The rib may be integral with the plurality of segments. Additionally, or alternatively, the articulation member comprises a polymer. The plurality of segments may be integral with one another, and each segment may be pivotable relative to the adjacent segment by a living hinge. Furthermore, an inner surface of each segment of the plurality of segments may define an arc between about 180 degrees and about 270 degrees. The at least one aperture of each segment may include a first aperture and a second aperture. The first apertures of the plurality of segments may be aligned with one another, and the second apertures of the plurality of segments may be aligned with one another. A first steering wire may extend through the first apertures and a second steering wire may extend through the second apertures. The shaft may include a sheath surrounding the articulation member and the working channel. The shaft may include a first channel and a second channel between the sheath and the articulation member, the rib may divide the first channel from the second channel. Additionally, in some examples, the medical device may comprise at least one electronic component distal to the plurality of segments. The at least one electronic component may be coupled to a control member extending through the first channel or the second channel. The at least one electronic component may include an imaging device or a light source, for example. In some aspects, each segment of the plurality of segments may be symmetric about a plane that includes the rib. In some examples, at least a portion of the articulation member is radiopaque. In some aspects, the rib may define a plurality of holes along a length of the rib. The medical device may further comprise a handle and the steering wire(s), the handle including an actuator operably coupled to the steering wire(s), wherein manipulating the actuator may tension the steering wire(s) extending from a proximal end to a distal end of the shaft.

The disclosure also includes a medical device with a shaft defining a working channel extending from a proximal end to a distal portion of the shaft. The distal portion of the shaft may include an articulation member comprising a plurality of segments radially outward of the working channel and a rib. Each segment may have an arc-shaped cross-section and define a first aperture and a second aperture for receiving steering wires therethrough. The first apertures of the plurality of segments may be aligned with one another and the second apertures of the plurality of segments may be aligned with one another. Each segment may be pivotable relative to an adjacent segment. The rib may extend along a longitudinal axis of the shaft, e.g., the rib extending radially outward from the plurality of segments in a direction away from the working channel. The articulation member may comprise a polymer, a metal, a metal alloy, or a combination thereof. According to some aspects, the rib may comprise a metal or metal alloy. The rib may define a plurality of holes along a length of the rib. A first steering wire member may extend through the first apertures and a second steering wire may extend through the second apertures.

The disclosure also includes a medical device with a shaft defining a working channel extending from a proximal end to a distal portion of the shaft. The distal portion of the shaft may include an electronic component and an articulation member proximal to the electronic component. The articulation member may comprise a plurality of segments radially outward of the working channel and a rib. Each segment may have an arc-shaped cross-section and define at least one aperture. The at least one aperture of each segment may be aligned with the at least one aperture of an adjacent segment for receiving a steering wire therethrough, and each segment may be pivotable relative to the adjacent segment. The rib may extend along a longitudinal axis of the shaft and be coupled to, or integral with, each segment of the plurality of segments, e.g., rib extending radially outward from the plurality of segments in a direction away from the working channel.

### BRIEF DESCRIPTION OF THE FIGURES

The accompanying drawings, which are incorporated in and constitute a part of this application, illustrate exemplary aspects that, together with the written description, explain the principles of this disclosure. The figures depict exemplary aspects according to this disclosure, as follows:

FIG. 1 depicts a side-view of an exemplary medical device, including a handle and a shaft, according to some aspects of this disclosure.

FIGS. 2A-2C illustrate features of an articulation member of the medical device of FIG. 1.

FIG. 3 depicts a cross-sectional view of the shaft of the medical device of FIG. 1.

FIG. 4 shows another exemplary articulation member, according to some aspects of this disclosure.

### DETAILED DESCRIPTION

Both the foregoing general description and the following detailed description are exemplary and explanatory only and are not restrictive of the features, as claimed. As used herein, the terms “comprises,” “comprising,” “having,” “including,” or other variations thereof, are intended to cover a non-exclusive inclusion such that a process, method, article, or apparatus that comprises a list of elements does not include only those elements but may include other elements not expressly listed or inherent to such a process, method, article, or apparatus. In this disclosure, relative terms, such as, for example, “about,” “substantially,” “generally,” and “approximately” are used to indicate a possible variation of ±10% in a stated value or characteristic.

Aspects of this disclosure are described with reference to exemplary medical devices with articulation members providing relatively more space to accommodate, for example, medical tools and connections to electronic components of the medical devices. The medical device may include a handle and a shaft that includes an articulation member and a working channel. The shaft may include at least one control member (e.g., pull wire) extending through the shaft, e.g., to control the articulation member. The articulation member may have an arc-shaped or noncircular cross-section, e.g., extending along only a portion of the inner circumference of the shaft. Additionally, the articulation member may provide for at least one channel between the articulation member and a sheath of the shaft for receiving, e.g., one or more electronic connections such as wires providing power and/or transferring data to and from electronic components of the shaft.

Reference will now be made in detail to examples to help illustrate aspects of the disclosure through the accompanying drawings. Wherever possible, the same reference numbers will be used throughout the drawings to refer to the same or like parts.

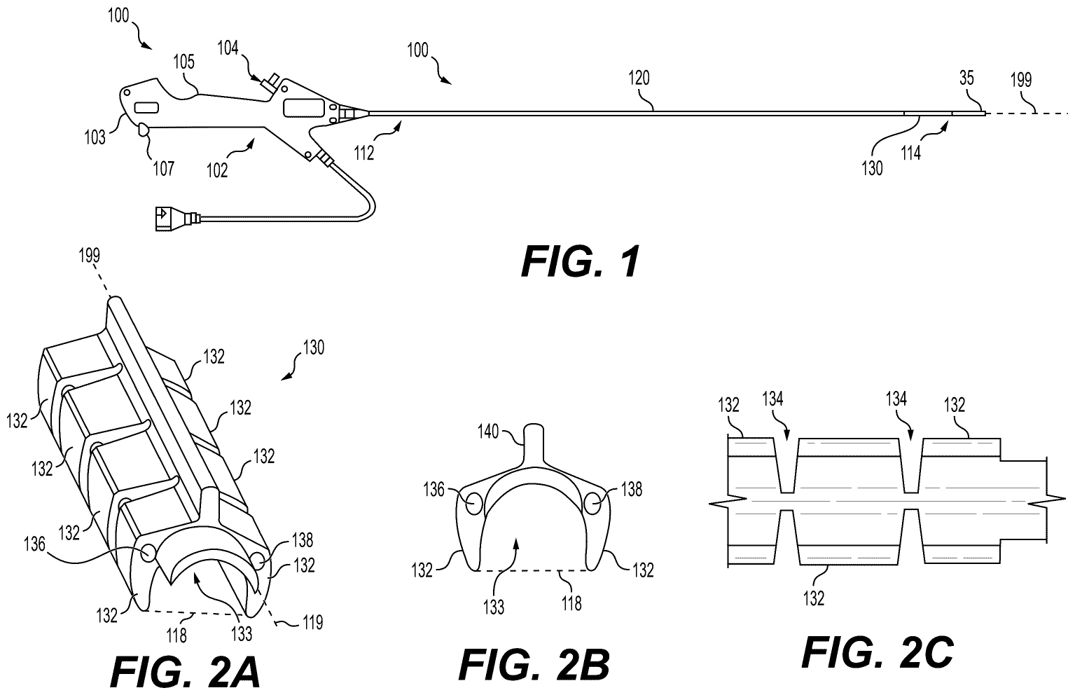

FIG. 1 depicts an exemplary medical device 100 according to some aspects of this disclosure, medical device 100 including a handle 102 and a shaft 120. The medical device 100 may be any suitable device configured to allow a user to access, view internal areas of a subject's body, and perform medical diagnoses and/or treatments on a subject. For example, the medical device 100 may be an ureteroscope, an endoscope, a hysteroscope, a bronchoscope, a cystoscope, or other scopes or similar medical devices.

The shaft 120 may extend from a proximal end 112 to a distal end 114. In some aspects, the distal end 114 or a portion of the shaft 120 proximate the distal end 114 may include one or more electronic components 35 such as, e.g., one or more light sources and/or imaging devices. The shaft 120 may include an end cap, for example, which includes the light source(s) and/or imaging device(s). The light source(s) may include any element capable of delivering light to a target site. Exemplary light sources include, for example, light emitting diode(s) and optical fibers. The imaging device(s) may include a camera and/or other optical device(s) capable of capturing an image of a target site.

An umbilicus may extend from the handle 102 (e.g., from a proximal portion of handle 102) and may be removably coupled (e.g., directly, or indirectly) to a processing unit. The processing unit may be configured to process information (e.g., sensor data, imaging data, light data, etc.) received from the medical device 100. In some aspects, the processing unit may be a controller associated with the medical device 100. Electronic components 35 such as light sources and/or imaging devices may be electrically coupled (e.g., directly, or indirectly) to the processing unit, for example, via electronic connections (e.g., one or more wires or cables) extending through handle 102 and/or shaft 120, and through the umbilicus.

The handle 102 may include a port 104 in fluid communication with a working channel 116 of shaft 120. Port 104 may be useful to introduce and/or remove medical tools, fluids, or other materials to and from the shaft 120 for delivery to a target site. The working channel 116 of shaft 120 may extend from the proximal end 112 to the distal end 114 of shaft 120, e.g., having a distal opening at the distal end 114 of shaft 120. Medical tools and instruments, such as biopsy forceps, graspers, baskets, snares, and/or other devices, may be inserted into the port 104 and advanced through the working channel 116 to exit through a distal opening of a distal end 114 of the shaft 120 into a subject's body to perform a medical procedure. The shaft 120 may provide access into a bodily lumen (e.g., along a tortuous path, for example, via a natural body orifice).

The handle 102 may include one or more actuators, e.g., actuator 107, to control articulation of the shaft 120. Exemplary actuators may include, e.g., knobs, thumbwheels, or levers. The actuator 107 may help steer the distal end 114 of the shaft 120, e.g., by manipulating an articulation member 130 of the shaft 120. The actuator 107 may be operably coupled to the articulation member 130 (e.g., directly, or indirectly) via one or more steering wires.

As mentioned above, the shaft 120 may include an articulation member 130, e.g., proximate distal end 114. The shaft 120 may include at least one steering wire operably coupled to the handle 102 and extending through the proximal end 112 of the shaft 120 and through the articulation member 130. The shaft 120 may include a sheath 155, the articulation member 130 and the working channel 116 being enclosed within the sheath 155. Optionally, the shaft 120 may include or be coupled to an end cap. For example, the end cap may be affixed, releasably attached, or otherwise coupled to a distal-most segment 132 of the articulation member 130.

FIGS. 2A-2B depict the articulation member 130. The articulation member 130 includes a rib 140 and a plurality of segments 132. The plurality of segments 132 may be symmetric about a plane that includes the rib 140. Each segment 132 of the plurality of segments 132 of the articulation member 130 may be pivotable relative to adjacent segments 132. The rib 140 may serve as an offset member or offset hinge of the articulation member 130.

In some aspects, the plurality of segments 132 is a single integral piece (e.g., FIGS. 2A-2C). In other aspects, the plurality of segments 132 includes segments 132 that are coupled together (see, e.g., FIG. 4). In either instance, rib 140 may be integral with one or more segments 132 or may be a separate component coupled to the plurality of segments 132.

In examples in which the plurality of segments 132 is integral to one another (e.g., the example shown in FIGS. 2A-2C), the segments 132 may be pivotable relative to one another via living hinges. The living hinges may be defined by a plurality of cutouts 134 or spaces between segments 132. In some examples, the cutouts 134 may have the same or substantially the same size and shape. On other examples, one or more cutouts 134 may have different sizes and/or shapes relative to other cutouts 134. The size and shape of cutouts and the living hinges may define the extent of bending between adjacent segments 132. For example, the cutouts 134 may be configured to help the shaft 120 of the medical device 100 bend at angles of 30, 60, or 90 degrees. Adjusting the pattern and spacing of the cutouts 134 may alter the shape of the articulation member 130. For example, a series of evenly spaced cutouts 134 may help provide a continuous bend that may form a linear shape of articulation member. Alternatively, the cutouts 134 may be irregularly spaced to enable the articulation member 130 to take on specific shapes.

As mentioned above, the plurality of segments 132 of the articulation member 130 may include an arc-shaped cross-section. For example, as shown in FIG. 2A, the inner surface of each of the plurality of segments 132 may define an arc less than 360 degrees, such as, e.g., an arc between about 150 degrees and about 300 degrees, between about 180 degrees and 270 degrees, or between about 210 degrees and about 240 degrees.

The plurality of segments 132 may be radially outward of the working channel 116 of the shaft 120. For example, the inner surfaces of the plurality of segments 132 may define the upper wall of a lumen 133 that receives the working channel 116 of the shaft 120. Ends of the plurality of segments 132 opposite the rib 140 may be spaced apart by a gap 118, which may be greater than the cross-sectional dimension of the working channel 116.

Each segment 132 of the plurality of segments 132 of the articulation member 130 may define at least one aperture for receiving a steering wire therethrough. In the example illustrated in FIGS. 2A-2B, each segment 132 defines two apertures, a first aperture 136 and a second aperture 138. The first apertures 136 may be at least partially or completely aligned in order to receive a first steering wire through the articulation member 130. Similarly, the second apertures 138 may be at least partially or completely aligned in order to receive a second steering wire through the articulation member 13 .In this type of example, the articulation member 130 may be configured for bi-directional steering of the shaft 120. Two steering wires passing, respectively, through the first apertures 136 and the second apertures 138, may provide for bending of the shaft 120 in two directions. The two steering wires may be controlled, e.g., tensioned, by the actuator 107 of the handle 102 to move the articulation member 130 in the different directions.

In other examples, the articulation member 130 may be configured for mono-directional movement, which may be suitable for medical procedures where bending in a single direction is sufficient. For example, each segment 132 may include only the first apertures 136 and one steering wire through the series of aligned first apertures 136.

The rib 140 may extend along a longitudinal axis 199 of the shaft 120. The rib 140 may provide axial stiffness to articulation member 130 and shaft 120. As shown, the rib 140 may extend radially outward from the plurality of segments 132 in a direction away from the lumen 133, that is, in a direction away from the working channel 116.

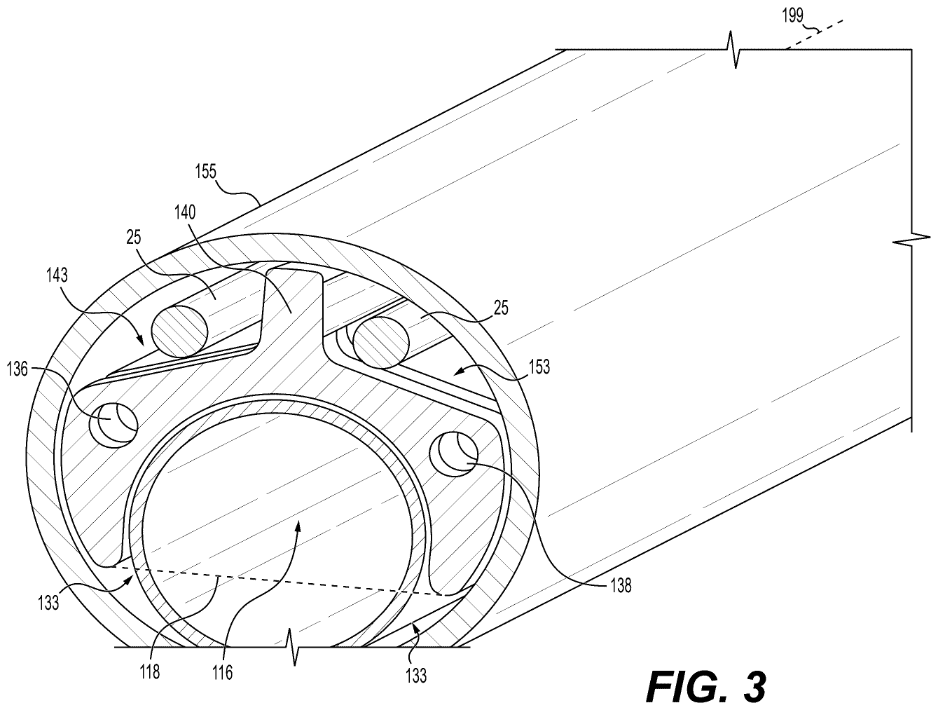

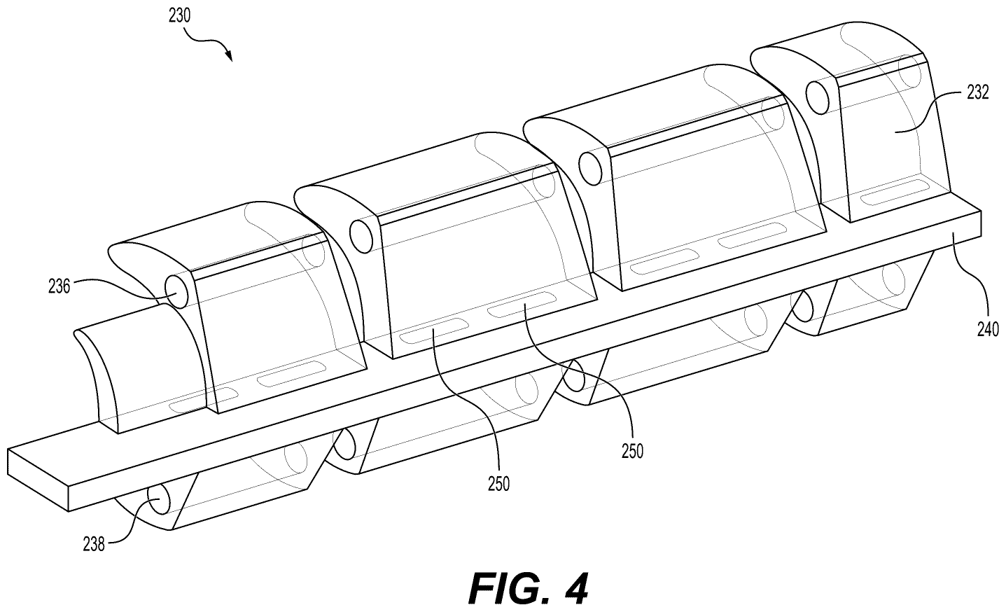

Aspects of this disclosure are further described with reference to FIG. 3, which shows a sheath 155 the shaft 120 surrounding the articulation member 130 and the working channel 116. The shaft 120 includes a first channel 143 and a second channel 153 between the sheath 155 and the articulation member 130. Each channel 143, 153 may be configured to accommodate electronic connections, fluidics, an active working channel, or other device components.

For example, each channel 143 and 153 may receive one or more control members 25, each of which may be an electronic connection. As discussed above, the medical device 100 may comprise at least one electronic component 35 (e.g., an imaging device and/or light source). For example, the electronic component(s) 35 may be distal to the plurality of segments 132 and may be coupled to control member(s) 25 (e.g., electronic connections) extending through the first channel 143 and/or the second channel 153. The control member(s) 25 may provide power and/or transfer data between the electronic component(s) 35 and the handle 102, optionally further to a processing unit via the umbilicus as discussed above. Although only one control member 25 is shown in each of the first channel 143 and the second channel 153, in some aspects, more than one control member 25 may be in the first channel 143 and/or the second channel 153.

The first channel 143 and the second channel 153 may be adjacent to each other, e.g., on opposite sides of the rib 140, between the sheath 155 and the articulation member 130. For example, the rib 140 may divide the first channel 143 from the second channel 153. Each of the first channel 143 and the second channel 153 may have an approximately triangular shape, although other shapes are contemplated.

In use, the first channel 143 and the second channel 153 may provide designated pathways for control members 25 and/or other components. For example, the plurality of control members 25 may be constrained into the first channel 143, the second channel 153, and the working channel 116. By assigning control members 25 to designated areas, each control member 25 may remain in a predefined area relative to the aperture(s) 136 or the aperture(s) 138. This allows the user of the medical device 100 to know or anticipate the positioning of each control member 25 relative to the device's steering mechanism during articulation or movement of the shaft 120. As a result, the user may quickly identify and manipulate the control members 25 as needed for medical procedures.

Additionally, during procedures, control members 25 may naturally move to positions of less resistance within a channel when actuated to complete their movement. The rib 140 acting as a divider may help to constrain the control members 25 within their designated channels (e.g., the first channel 143 or the second channel 153) to avoid interference with each other.

FIG. 4 illustrates another exemplary articulation member 230 according to further aspects of this disclosure. The articulation member 230 may include any of the features of the articulation member 230 unless otherwise specified and may be used in connection to any of the features of medical device 100. The articulation member 230 includes a plurality of segments 232 and a rib 240. Each segment 232 may define two apertures, a first aperture 236 and a second aperture 238 similar to the first aperture 136 and the second aperture 138. In this example, the plurality of segments 232 are coupled together, rather than integral with one another. For example, the articulation member 230 includes a plurality of separate segments 232, akin to a vertebra, and a singular bending component in the form of the rib 240 placed on a neutral plane of the articulation member 230. The rib 240 is coupled to the plurality of segments 232.

According to some aspects, the plurality of segments 232 and the rib 240 comprise different materials. For example, the rib 140 may comprise a metal or metal alloy, while the plurality of segments 232 comprise a polymer. In another example, the rib 240 may comprise a polymer, and the plurality of segments 232 may comprise a polymer different than the polymer of the rib 240. As illustrated in FIG. 4, the rib 240 may include a series of holes 250 along its length. The holes 250 may be configured to engage with corresponding features on the segments 232, such as protrusions or tabs.

According to some aspects, at least a portion of the articulation member 130 or 230 may comprise a radiopaque material, e.g., to improve visibility under fluoroscopy or X-ray imaging. For example, the rib 140 or 240 may be laminated with a radiopaque material or may comprise a radiopaque filler. Additionally, or alternatively, at least a portion of the plurality of segments 132 or 232 may comprise a radiopaque material. The articulation member 130 or 230 may be manufactured via molding, or additive manufacturing, for example.

While principles of the disclosure are described herein with reference to illustrative aspects for particular medical uses and procedures, the disclosure is not limited thereto. Those having ordinary skill in the art and access to the teachings provided herein will recognize additional modifications, applications, aspects, and substitution of equivalents all fall in the scope of the aspects described herein. Accordingly, the disclosure is not to be considered as limited by the foregoing description.

What is claimed is:

1. A medical device comprising:
a shaft defining a working channel extending from a proximal end to a distal portion of the shaft, wherein the distal portion of the shaft includes an articulation member comprising:
a plurality of segments radially outward of the working channel, each segment having an arc-shaped cross-section and defining at least one aperture, the at least one aperture of each segment being aligned with the at least one aperture of an adjacent segment for receiving a steering wire therethrough, and each segment being pivotable relative to the adjacent segment; and
a rib extending along a longitudinal axis of the shaft and coupled to, or integral with, each segment of the plurality of segments, the rib extending radially outward from the plurality of segments in a direction away from the working channel.

2. The medical device of claim 1, wherein the rib is coupled to the plurality of segments, and wherein the rib and the plurality of segments comprise different materials.

3. The medical device of claim 1, wherein the rib comprises a metal or metal alloy.

4. The medical device of claim 1, wherein the rib is integral with the plurality of segments, the articulation member comprising a polymer.

5. The medical device of claim 1, wherein the plurality of segments is integral with one another, each segment being pivotable relative to the adjacent segment by a living hinge.

6. The medical device of claim 1, wherein an inner surface of each segment of the plurality of segments defines an arc between about 180 degrees and about 270 degrees.

7. The medical device of claim 1, wherein the at least one aperture of each segment includes a first aperture and a second aperture, the first apertures of the plurality of segments being aligned with one another, and the second apertures of the plurality of segments being aligned with one another.

8. The medical device of claim 7, further comprising a first steering wire extending through the first apertures and a second steering wire extending through the second apertures.

9. The medical device of claim 1, wherein the shaft includes a sheath surrounding the articulation member and the working channel, and wherein the shaft includes a first channel and a second channel between the sheath and the articulation member, the rib dividing the first channel from the second channel.

10. The medical device of claim 9, wherein the medical device comprises at least one electronic component distal to the plurality of segments, the at least one electronic component being coupled to a control member extending through the first channel or the second channel.

11. The medical device of claim 10, wherein the at least one electronic component includes an imaging device or a light source.

12. The medical device of claim 1, wherein each segment of the plurality of segments is symmetric about a plane that includes the rib.

13. The medical device of claim 1, wherein at least a portion of the articulation member is radiopaque.

14. The medical device of claim 1, wherein the rib defines a plurality of holes along a length of the rib.

15. The medical device of claim 1, further comprising the steering wire and a handle with an actuator operably coupled to the steering wire, wherein manipulating the actuator tensions the steering wire extending from a proximal end to a distal end of the shaft.

16. A medical device comprising:
a shaft defining a working channel extending from a proximal end to a distal portion of the shaft, wherein the distal portion of the shaft includes an articulation member comprising:
a plurality of segments radially outward of the working channel, each segment having an arc-shaped cross-section and defining a first aperture and a second aperture for receiving steering wires therethrough, wherein the first apertures of the plurality of segments are aligned with one another and the second apertures of the plurality of segments are aligned with one another, and wherein each segment is pivotable relative to an adjacent segment; and
a rib extending along a longitudinal axis of the shaft, the rib extending radially outward from the plurality of segments in a direction away from the working channel;
wherein the articulation member comprises a polymer, a metal, a metal alloy, or a combination thereof.

17. The medical device of claim 16, wherein the rib comprises a metal or metal alloy.

18. The medical device of claim 16, wherein the rib defines a plurality of holes along a length of the rib.

19. The medical device of claim 16, further comprising a first steering wire member extending through the first apertures and a second steering wire extending through the second apertures.

20. A medical device comprising:
a shaft defining a working channel extending from a proximal end to a distal portion of the shaft, wherein the distal portion of the shaft includes an electronic component and an articulation member proximal to the electronic component, the articulation member comprising:
a plurality of segments radially outward of the working channel, each segment having an arc-shaped cross-section and defining at least one aperture, the at least one aperture of each segment being aligned with the at least one aperture of an adjacent segment for receiving a steering wire therethrough, and each segment being pivotable relative to the adjacent segment; and
a rib extending along a longitudinal axis of the shaft and coupled to, or integral with, each segment of the plurality of segments, the rib extending radially outward from the plurality of segments in a direction away from the working channel;
wherein the shaft includes a sheath surrounding the articulation member and the working channel, and wherein the shaft includes a first channel and a second channel between the sheath and the articulation member, the rib dividing the first channel from the second channel.
